(19) United States
(12) Patent Application Publication
(10) Pub. No.: US 2006/0078573 A1
(43) Pub. Date: Apr. 13, 2006

(54) Methods of modifying crystal habit

(76) Inventors: Theodore Randolph, Niwot, CO (US); Corinne Lengsfeld, Denver, CO (US); Daniel Jarmer, Westfield, NJ (US)

Correspondence Address: SHERIDAN ROSS PC, 1560 BROADWAY, SUITE 1200, DENVER, CO, 80202, US

(21) Appl. No.: 11/152,041

(22) Filed: Jun. 13, 2005

Related U.S. Application Data
(60) Provisional application No. 60/578,967, filed on Jun. 11, 2004.

(51) Int. Cl. A61K 9/00

(52) U.S. Cl. 424/400

(57) ABSTRACT

The invention provides methods of modifying the crystal habit of a compound without altering the crystal structure of the compound through a controlled precipitation technique in the presence of a crystal growth inhibitor as well as the crystallized compounds formed by these methods. Using these methods, the crystal habit of the compound may be modified from acicular to bipyramidal. The modification in crystal habit is attributable to a preferential adsorption mechanism of the crystal growth inhibitor to a fast growing crystal face of the compound. Powder flow properties of the crystallized product are significantly enhanced with the habit modification. This selective crystal habit modification using a crystal growth inhibitor provides a strategy to circumvent the manufacturing difficulties associated with acicular crystal habits, and may increase the manufacturing capability of supercritical fluid based crystallization and precipitation technologies.

### CROSS REFERENCE TO RELATED APPLICATION

This application claims priority under 35 U.S.C. § 119(e) to U.S. Provisional Patent Application Ser. No. 60/578,967 filed Jun. 11, 2004, which is incorporated herein in its entirety by this reference.

### FIELD OF THE INVENTION

The invention relates to a method of modifying crystal habit during precipitation, and more specifically methods of selectively modifying crystal habit during precipitation with a compressed fluid antisolvent.

### BACKGROUND OF THE INVENTION

Most active pharmaceutical ingredients (APIs) are administered as solid dosage forms produced by the formulation and processing of powdered solids. The success or failure of these formulations is often dependent upon the physical properties of the API since the physical properties affect powder flow, bulk handling, ease of compression, and physical stability. Crystal habit and the crystal size distribution are two key physical properties involved in the formulation of solid dosage forms. Thus, control over these properties during solution crystallization is important in determining the success of a formulation.

Precipitation with a Compressed-fluid Antisolvent, or PCA, involves the precipitation (or crystallization) of a solute from an organic solvent by the addition of a compressed gas, which acts as an antisolvent for the solute. Two benefits often associated with PCA include single step processing of particulate pharmaceuticals with controlled characteristics, and the efficient separation (by decompression) of the antisolvent from both the solvent and solid products. When PCA is conducted above the mixture critical point (i.e. complete miscibility between the solvent and antisolvent), the precipitation kinetics and resulting product quality can be determined by the rate of mixing between two initially separate fluid streams. In order to minimize the effect of imperfect mixing on the precipitation kinetics, the characteristic times for mixing (i.e. macromixing, mesomixing, and micromixing) must be less than the characteristic times for particle nucleation and growth. This requirement is being met through the design and development of injectors that: (1) produce a region of high turbulent energy dissipation (i.e. high intensity mixing), and (2) ensure that both process streams pass through the region of high intensity mixing without bypassing.

A consequence of the fast mixing between the two process streams is a rapid crystallization, which often results in a crystal habit that is acicular (needle-shaped) or plate-like (platy or flaky). These crystal habits are a result of the very fast crystal growth rate (i.e. high supersaturation level) that is obtained within the injector, or immediately downstream of the injector, where supersaturated effluent can enter a particle collection vessel. However, acicular and plate-like crystals habits are disfavored in product manufacturing because they have poor powder flow properties and filtration characteristics, and they have a tendency to cake, and are often brittle. Brittle particles often fracture upon handling, which may result in a polydisperse particle size distribution (PSD). Polydisperse PSDs are unfavorable since they adversely affect powder mixing phenomena, provide poor content uniformity, and afford the possibility of particle segregation in mixed materials. Furthermore, pharmaceutical powders with an acicular or plate-like habit are typically cohesive and characterized by a high compressibility. A high compressibility is indicative of a powder that is non-free flowing, which makes product tableting difficult and inefficient. Overall, crystals with these habits may require additional processing steps (e.g. fluid energy milling followed by size classification) in order to achieve the required PSD for a particular formulation. The addition of subsequent processing steps reduces the processing advantages of PCA, and may render PCA as a nonviable manufacturing technique for some materials.

There are several processing strategies that can be used to modify crystal habit, and thus circumvent the production of unfavorable crystal habits that render drug formulation difficult. For example, it is well known that crystal habit maybe modified by operating a crystallizer under different levels of supersaturation, crystallizing the solute from different solvents, changing the process temperature, or adding a growth inhibitor to selectively modify crystal habit. The literature is replete with discussions on these topics for conventional solution crystallization. Similar to conventional crystallization, changes in the process temperature and the process solvent have resulted in a change in crystal habit during PCA. But these changes often result in the production of a new polymorph with different physical and chemical properties, which may be unacceptable in the development of a drug formulation. There have been no reports concerning the use of additives as growth inhibitors to selectively modify crystal habit during PCA. This form of habit modification is unique since crystal habit can be modified without changing the process temperature or pressure (i.e. phase behavior), which often results in the formation of a different polymorph with different physical properties.

Shekunov at el. (Crystal Growth and Design 3:603-10(2003)) reported the use of structurally similar molecules to alter crystal structure (hence crystal habit) during PCA, but this technique of modifying the crystal structure resulted in a product with different solid state properties. Additionally, U.S. patent application 20020114844 to Hanna et al. describes a method of ‘coating’ crystals with additives using PCA. But as shown by the examples in the patent application, this method does not modify the crystal habit of the solute.

Thus, a method of modifying and controlling crystal habit during PCA is desired. Preferably, the process would allow for the modification of crystal habit while preserving the original crystal structure and thereby retaining the same physical and chemical properties of a crystallized API.

### SUMMARY OF THE INVENTION

The present invention provides methods of selectively modifying crystal habit through the use of a growth inhibitor when an active pharmaceutical ingredient (API) is processed using precipitation with a compressed-fluid antisolvent (PCA). These methods produce a crystal habit that is more suitable for product manufacturing, while retaining the same crystal structure and physical properties of the original API. In these methods, the API is simultaneously precipitated with a crystal growth inhibitor. This is accomplished through rapid mixing of the solvent and antisolvent process streams. Preferably the rapid mixing is conducted with an injector with a confined mixing chamber.

One embodiment of the present invention is a method that includes contacting a solvent that contains a compound and a crystal growth inhibitor, with an anti-solvent to extract the solvent from a co-precipitate of compound and the crystal growth inhibitor. Using this method, the crystal habit of the compound is modified without altering the crystal structure of the compound. The contacting may be conducted in a confined mixing chamber, and the co-precipitate may be discharged into a particle collection vessel.

In preferred embodiments, the solvent is an organic solvent such as methylene chloride, methanol, acetone, acetonitrile, methyl ethyl ketone, isopropanol, propanol, butanol, ether, benzene, hexane, hexanol, ethanol, cyclohexane, isooctane the anti-solvent is a fluid such as carbon dioxide, nitrogen, nitrous oxide, sulphur hexafluoride, xenon, ethane, ethylene, chlorotrifluoromethane, chlorodifluoromethane, dichloromethane, trifluoromethane, helium, neon, and mixtures thereof.

The anti-solvents of the invention are preferably a supercritical fluid or at least a near-supercritical fluid, and may include a co-solvent such as water, methanol, ethanol, isopropanol, acetone and combinations thereof. Preferably, the anti-solvent is present in excess to the solvent during the contacting step of these methods.

The compounds of the invention may be pharmaceutical compounds, and preferably pharmaceutical compounds intended for formulation in dosage formulations formed from dry powders.

The crystal growth inhibitors of the invention are typically hydrophobic chemicals, and preferably polyanhydrides such as poly (sebacic anhydride).

The methods of the invention may be conducted using a mass ratio of the crystal growth inhibitor to the compound of less than about 1:1, and preferably about 1:5. These methods may also be conducted using a solvent having a total solids concentration between about 0.5 wt % and about 1.5 wt %.

One embodiment is an article of manufacture containing a co-precipitate of a crystalline compound and a crystal growth inhibitor. Preferably, the article of manufacture is a pharmaceutical formulation containing a co-precipitate of a crystalline compound and a crystal growth inhibitor. In one preferred embodiment, the pharmaceutical formulation contains a co-precipitate of griseofulvin and poly (sebacic anhydride).

One embodiment of the invention is a co-precipitate of a crystalline compound and a crystal growth inhibitor formed by contacting a solvent containing a compound and a crystal growth inhibitor, with an anti-solvent to extract the solvent from a co-precipitate of the compound and the crystal growth inhibitor.

### BRIEF DESCRIPTION OF THE DRAWINGS

FIG. 1 a is an SEM micrograph of bulk griseofulvin particles obtained from the reagent bottle, and FIG. 1b is an SEM micrograph of pure griseofulvin particles crystallized using PCA under the same conditions as the drug-polymer mixtures.

FIGS. 2a and 2b show TEM images of a PSA-griseofulvin particle produced during PCA. The mass ratio of PSA/griseofulvin was 1:39, and the total solids concentration in the feed was 0.75 wt % for the particle shown in FIG. 2a, and 1.5 wt % for the.particle shown in FIG. 2b.

FIG. 3 shows the differential number distributions of particles produced during PCA. The total feed concentration was 0.75 wt %. The mass ratios of PSA/griseofulvin in the feed were: (□) 1:39, (◯) 1:19, (▴) 1:5.

FIG. 4 shows a differential number distributions of particles produced during PCA. The total feed concentrations were (▴) 0.75 wt % and (◯) 1.5 wt %. The mass ratio of PSA/griseofulvin was 1:39.

FIGS. 5a and 5b show X-ray powder diffraction (XRPD) spectra of bulk griseofulvin, and PSA homopolymer, respectively.

FIGS. 6a and 6b show X-ray powder diffraction (XRPD) spectra of PCA processed particles in which the mass ratios of PSA/griseofulvin were 1:1 and 1:5, respectively.

FIGS. 7a and 7b show X-ray powder diffraction (XRPD) spectra of PCA processed particles, in which the mass ratios of PSA/griseofulvin were 1:19, and 1:39, respectively.

FIG. 8 shows DSC thermograms obtained for selected PSA-griseofulvin samples prepared by the methods of the present invention.

FIG. 9 shows a dissolution profile for a PSA-griseofulvin sample produced by the methods of the present invention. The dissolution medium was a USP-simulated gastric fluid containing 2.0 wt % SDS. The data points shown are the mean of triplicate experiments and the error bars represent one standard deviation of the mean.

FIGS. 10a and 10b shows XRPD spectra of bulk griseofulvin particles after storage at 25° C./60% RH for 23 days or 40° C. 70% RH for 23 days, respectively.

FIGS. 11a and 11b show XRPD spectra of PSA-griseofulvin particles after storage at 25° C./60% RH for 23 days or 40° C. 70% RH for 23 days, respectively. The mass ratio of PSA/griseofulvin in the feed was 1:5.

### DETAILED DESCRIPTION OF THE INVENTION

The present invention is drawn to a process of enhancing the powder flow properties of a crystallized compound by modifying the crystal habit of the compound without altering the crystalline structure. This method of selective crystal habit modification includes the crystallization of the compound in the presence of a crystal growth inhibitor and circumvents the manufacturing difficulties associated with acicular crystal habits, thereby increasing the manufacturing capability of crystallization and precipitation technologies.

The methods of selectively modifying the crystal habit of a compound without altering the crystal structure include contacting a solvent comprising a compound and a crystal growth inhibitor, with an anti-solvent fluid to coprecipitate the compound and the crystal growth inhibitor. The crystallization of the compound in the presence of the crystal growth inhibitor modifies the crystal habit of the compound without altering the crystal structure of the compound. In a preferred embodiment, the contact is conducted in a confined mixing chamber to promote the rapid precipitation of the compound and the crystal growth inhibitor. The precipitating crystal particles may then be discharged into a particle collection vessel separate from the confined mixing chamber.

The solvent used in the methods of the invention may be any liquid or gas in which the compound may be solubilized. The solvent may be a single, pure liquid or a mixture of multiple liquids having the desired physical and chemical characteristics. Preferably, the solvent used is an organic solvent. An exemplary organic solvent is methylene chloride. The solvent is chosen to be miscible with the anti-solvent in all proportions under the operating conditions employed in these methods. This miscibility assures that the contacting of the solvent and anti-solvent results in dissolution of the fluids in one another, precipitating the compound and the crystal growth inhibitor. Preferably, the solvent and anti-solvent are totally miscible in all proportions under the operating conditions used in contacting the solvent with the anti-solvent.

The anti-solvent used in these methods may be any fluid, such as a liquid or a gas, or mixture of fluids which effectively acts as an anti-solvent for the chosen solvent, allowing efficient separation of the anti-solvent (by decompression) from both the solvent and the solid precipitate. Thus, the anti-solvent is chosen to combine with the solvent such that the compound and the crystal growth inhibitor are insoluble or substantially insoluble in the mixture. Preferably, the anti-solvent fluid is a supercritical or near-critical fluid under the operating conditions used in the contacting step of the methods of the invention. Thus, the anti-solvent fluid or fluids are preferably at or above the critical pressure and critical temperature, simultaneously. In practice, the pressure of the fluid is typically in the range of between about 10 bar and about 250 bar, and preferably about 85 bar and the temperature is maintained in the range of between about 0° C. and about 100° C., and preferably about 35° C. However, some fluids (eg, helium and neon) have particularly low critical pressures and temperatures, and may need to be used under operating conditions well in excess of (such as up to 200 times) those critical values.

Near-critical anti-solvent fluids include high pressure liquids, which are fluids at or above their critical pressure but below (and preferably close to) their critical temperature, as well as dense vapors, which are fluids at or above their critical temperature but below (and preferably close to) their critical pressure.

Preferably, the anti-solvent is a supercritical fluid such as supercritical carbon dioxide, nitrogen, nitrous oxide, sulphur hexafluoride, xenon, ethane, ethylene, chlorotrifluoromethane, chlorodifluoromethane, dichloromethane, trifluoromethane or a noble gas such as helium or neon, or a supercritical mixture of any of these. Most preferably the anti-solvent is supercritical carbon dioxide.

The anti-solvent fluid may contain one or more modifiers or co-solvents. These modifiers may be used to change the intrinsic properties of that fluid in or around its critical point, and in particular, change the anti-solvent's ability to dissolve other materials. Suitable modifiers include water, methanol, ethanol, isopropanol and/or acetone. Preferably, any modifier present in the anti-solvent constitutes less than about 40 mole %, and more preferably less than about 20 mole %, and most preferably between about 1 mole % and about 10 mole %, of the anti-solvent fluid.

The compound present in the solvent may be any compound that will precipitate under the conditions employed in the methods of the present invention. The compound is typically a compound that is targeted for handling and/or storage as a crystalline precipitate and for which control over the bulk properties of the crystalline powder is therefore important. Preferably, the compound is a pharmaceutical compound having a therapeutic activity in a mammal. Particularly suitable pharmaceutical compounds include those compounds typically administered in an oral dosage formulation that requires powder processing of the compound to make the desired dosage formulation. Such dosage formulation will include tablets, capsules, powders and the like. An exemplary compound is the oral antifungal compound, griseofulvin which has a very low aqueous solubility. This low aqueous solubility results in a slow dissolution rate coupled with an erratic and incomplete absorption profile. When griseofulvin is crystallized from methylene chloride solutions using compressed carbon dioxide as the antisolvent, the compound is known to crystallize in an acicular habit. However, when crystallized using the methods of the present invention, griseofulvin crystallizes in a bipyramidal habit having more desirable powder flow and processing characteristics and a more consistent dissolution profile without alteration of the crystal structure and the storage stability of the drug.

The crystal growth inhibitor may be any chemical that, when co-precipitated with the compound in the methods of the invention acts to modify the crystal habit of the compound without altering the crystal structure of the compound precipitate. The crystal growth inhibitor is chosen based upon its molecular structure, solubility in the process solvent, lack of solubility in the anti-solvent, crystallinity and hydrophobicity. Preferably, the crystal growth inhibitor is a hydrophobic chemical that is soluble in an organic solvent and insoluble in carbon dioxide. Polyanhydrides are particularly suitable crystal growth inhibitors for use in the methods of the present invention, and an exemplary crystal growth inhibitor is the hydrophobic, semi-crystalline polymer poly (sebacic anhydride) (PSA). PSA possesses several features which make it an attractive pharmaceutical additive. For example, polyanhydrides are biodegradable, nontoxic, and the hydrolytically-labile anhydride linkages degrade rapidly to form non-toxic diacid monomers which are eliminated from the body within weeks. Furthermore, polyanhydrides are generally considered to be safe as many anhydrides are natural constituents or metabolites of the human body.

The mass ratio of the crystal growth inhibitor to the compound in the solvent feed can affect the particle size distributions of the co-precipitate formed in these methods. Preferably, the mass ratio of the crystal growth inhibitor to the compound in the solvent feed is equal to or less than about 1:1, and is preferably about 1:1, and more preferably about 1:5 and more preferably about 1:19. Additionally, the total solids concentration in the solvent feed to the injector may affect the particle size distributions of the co-precipitate formed in these methods. Preferably, the total solids concentration in the feed may be between about 0.50 wt % and about 1.5 wt %, and preferably about 0.75 wt %, and more preferably about 0.9 wt %.

Bimodal particle size distributions are produced when the mass ratio of crystal growth inhibitor/compound in the feed is greater than about 1:1. In addition to the obvious situation where the crystal growth inhibitor and compound particles are physically segregated in the precipitated product, there are several other explanations for the observed bimodal particle size distributions. For example, decreasing the crystal growth inhibitor concentration in the feed (i.e. lower crystal growth inhibitor supersaturation ratio) will delay crystal growth inhibitor precipitation, allowing more time for compound crystals to grow uninhibited. Secondly, decreasing the crystal growth inhibitor concentration in the feed (with a concomitant increase in the concentration of the compound) provides less crystal growth inhibitor to act as a growth inhibitor and hence more compound crystals can grow to a larger size.

The concentration of the compound and the crystal growth inhibitor in the solvent must be chosen to give the desired ratio in the final co-precipitate. Preferably, this ratio is chosen such that the compound precipitates in a crystalline form under the operating conditions used while minimizing the amount of crystal growth inhibitor present that is still capable of modifying the crystal habit of the precipitated compound to display the desired physical and chemical characteristics.

As well as the relative concentrations of the compound and the crystal growth inhibitor, other parameters may be varied to achieve a co-precipitate having a desirable crystal habit. Such parameters include the temperature and pressure used in the contacting of the solvent and anti-solvent, and the flow rates of the solvent and anti-solvent upon contact with one another.

The contacting of the solvent and the anti-solvent is conducted in a manner that results in the simultaneous precipitation of the compound and the crystal growth inhibitor. Preferably, the solvent and the anti-solvent are rapidly mixed in a confined mixing chamber, and more preferably, the solvent and the anti-solvent are mixed by injection through an injection nozzle. In a preferred embodiment, the mixing is provided by introducing the solvent (containing the compound and the crystal growth inhibitor) and the anti-solvent through an injector within a confined mixing chamber. The mixing is conducted rapidly, and in less time than the time required for particle nucleation and growth, in order to minimize the effect of imperfect mixing on the precipitation kinetics.

The contacting rate will generally be chosen to ensure an excess of the anti-solvent with the solvent, to minimize the risk of the solvent re-dissolving and/or agglomerating the co-precipitate of compound and crystal growth inhibitor that is formed. At the point of contacting the solvent typically constitutes less than about 80 mole %, and preferably less than about 50 mole %, and more preferably less than about 30 mole %, and more preferably less than about 20 mole %, and most preferably less than about 5 mole % of the fluid mixture formed.

Preferably, the crystal habit of the compound is modified from acicular (needle like) to bipyramidal (i.e. platy or flaky) by the co-precipitation with the crystal growth inhibitor according to the methods of the present invention. Without intending to be bound by any single theory, it is believed that the crystal growth inhibitor preferentially adsorbs to some extent on a crystal face of the compound to decrease the crystal growth rate on that face. The adsorption of such a chemical to a crystal surface may block a kink site, and prevent or disrupt the subsequent bonding of additional solute molecules to the crystal lattice. SEM and TEM images of particles produced in accordance with the methods of this invention support this preferential adsorption mechanism and therefore, if the crystalline growth rate of the compound decreases due to preferential adsorption of crystal growth inhibitor to the crystal surface, the compound nucleation rate should have to increase in order to achieve an overall mass balance with the crystal growth inhibitor. An increased nucleation rate should favor the production of a larger number of smaller particles, resulting in a shift in the particle size distribution of the co-precipitate towards smaller size fractions. Doubling the feed concentration to the injector, for example from 0.75 wt % to 1.5 wt % (while maintaining a constant crystal growth inhibitor/compound mass ratio) may magnify this affect, shifting the co-precipitate particles towards a smaller size fraction.

The co-precipitate particles formed by the methods of the present invention are not homogeneous mixtures or mixed crystals of the two chemicals, but rather crystalline particles of the compound having the same crystalline structure expected of crystals formed from the crystallization of the compound in the absence of a crystal growth inhibitor, but having an altered crystal habit. These crystalline compound particles have thin coating of crystal growth inhibitor on one or more surfaces of the crystalline particle but do not incorporate the crystal growth inhibitor within or throughout the crystalline particles. Thus, an embodiment of the invention is a particulate co-precipitate of a crystal growth inhibitor and a compound of the types described above, in which the crystalline particles of the compound have a modified crystal habit and these crystalline compound particles may have a coating of a crystal growth inhibitor on one or more crystalline surface(s). Preferably, the crystal habit is a bipyramidal habit. Additionally, the crystalline co-precipitates of the invention preferentially display at least one desirable physical property such as improved powder flow, simplified bulk handling, ease of compression, enhanced physical stability, greater or more rapid dissolution and, in the case of an active pharmaceutical compound co-precipitate, elevated bioavailability.

Additional objects, advantages, and novel features of this invention will become apparent to those skilled in the art upon examination of the following examples thereof, which are not intended to be limiting.

### EXAMPLES

The model API used to demonstrate the methods of the present invention in the following examples was griseofulvin. Griseofulvin is an oral antifungal agent with a very low aqueous solubility which results in a slow dissolution rate coupled with an erratic and incomplete absorption profile. Unless a growth inhibitor is present, griseofulvin will crystallize in an acicular crystal habit from methylene chloride solutions when compressed CO₂ is used as the antisolvent.

### Example 1

### Synthesis of the Crystal Growth Inhibitor

A linear aliphatic polyanhydride was synthesized by a melt-polycondensation of acetyl terminated anhydride prepolymers. The synthesis of the acetylated prepolymer was performed using a modification of a previously reported procedure (Tarcha, P. et al., Journal of polymer Science: Part A: Polymer Chemistry 39:4189-95 (2001)). Glacial acetic acid (5.5 g) and triethylamine (9.8 g) were dissolved in methylene chloride (CH₂Cl₂) (25 mL), and the mixture was simultaneously stirred and purged with nitrogen for 30 minutes at 0° C. A 1:1 solution of sebacoyl chloride and CH₂Cl₂ (9 mL) was then added dropwise to the solution over a fifteen minute time interval. Stirring was continued for 4 hours at 0° C., followed by vacuum filtration for removal of the precipitated triethyl ammonium chloride. The filtrate was then washed sequentially with a saturated sodium bicarbonate (NaHCO₃) (100 mL×2) and distilled H₂O (50 mL×2), and then dried over sodium sulfate (Na₂SO₃). The Na₂SO₃ was removed via vacuum filtration, and the prepolymer solution was evaporated to dryness in a rotary vacuum evaporator at 25° C. The resulting residue was dried under vacuum for 4 hours and then stored at −20° C.

Homopolymers were synthesized by melt-polycondensation of the prepolymers at 180° C. and under vacuum (<0.1 mmHg) for 90 minutes using the method. In a typical polymerization, sebacic anhydride prepolymer (1.0 g) was charged to a 10 mL round bottom flask equipped with a vacuum fitting. The flask was immersed in a silicon oil bath at 180° C. After the prepolymers had melted (1 minute), high vacuum was applied to the flask, and the polymerization was allowed to proceed for 90 minutes under continuous agitation of the melt. Vigorous agitation of the melt was performed for 30 seconds every 15 minutes. The condensation product (acetic anhydride, poly(sebacic anhydride) (PSA)) was collected in a liquid nitrogen trap. Crude polymer was then purified by precipitation in petroleum ether from a CH₂Cl₂ solution. The polymer was dissolved in CH₂Cl₂ and precipitated dropwise in petroleum ether and isolated via vacuum filtration. The purified polymer was then stored in a moisture free environment at −20° C. to prevent hydrolysis.

### Example 2

### Effect of Feed Concentration and Mass Ratio on PCA Processing of Griseoftilvin

Griseofulvin was crystallized via PCA in the presence of the PSA while varying the feed concentration to the injector as well as the mass ratio of PSA to griseofulvin in the feed. The total solids concentration in the feed was either 0.75 or 1.5 wt %. The mass ratio of PSA to griseofulvin in the feed was either 5:1 or 1:39. Solutions of PSA in CH₂Cl₂, or PSA and griseofulvin in CH₂Cl₂, were processed using PCA. The process consisted of rapidly mixing a solution phase with an antisolvent (supercritical CO₂) inside a confined mixing chamber to promote rapid precipitation of both species. Particles were precipitated within the confined mixing chamber and then discharged into a particle collection vessel. The process operating temperature was maintained at 35° C., and the pressure was fixed at 85 bar in the particle collection vessel. The volumetric flow rate ratio of CO₂ to the solution was kept constant at 25:1 for all experimental runs. The solution flow rate was 1.0×10⁻² L min⁻¹ and the CO₂ flow rate (in the liquid state) was 2.5×10⁻² L min⁻¹. When sample lots >500 mg were required for physical characterization, the solution (about 50 mL) was charged to a separate high pressure pump and fed directly to the injector. For sample lots <100 mg the solution (about 10 mL) was charged to a high pressure sample cylinder fitted with a moveable piston prior to being fed to the injector. Control experiments were performed in which pure griseofulvin and pure PSA were processed under the same conditions as the drug-polymer mixtures. The effects of additional PSA to griseofulvin ratios, e.g. 1:1, 1:5, and 1:19, were investigated with a total solids concentration of 0.75 wt % in the feed. Furthermore, in order to investigate the effect of polymer molecular weight on crystal habit, a probe experiment was conducted with a mass ratio of PSA prepolymer to griseofulvin of 1:1.2, with a total solids concentration of 0.9 wt % in the feed.

### Example 3

### Characterization of the Crystal Growth Inhibitor and the Powder Product

¹-HNMR spectra were collected on a Varian Inova-500 (500-MHz) spectrometer using deuterated chloroform as the solvent. ¹H-NMR spectra of the acetylated sebacic acid prepolymer and the poly (sebacic anhydride) homopolymer showed the degree of oligomerization of the prepolymer from the integration ratio of the repeating unit (8 H, sebacic acid) at 1.3 ppm and the methyl terminal's peak of the anhydride end group at 2.2 ppm. Estimates of the average molecular weight of both the prepolymer and the polymer were made by determining the degree of polymerization. Table 1 lists the melting point (mp), degree of polymerization, calculated molecular weight (number average molecular weight as determined from the 1^{H}-NMR data), and IR characteristics for each polymer.

TABLE 1

| | mp | Mₙ | Degree of | IR |
|---|---|---|---|---|
| Material | [° C.] | [g/mol] | polymerization | [cm⁻¹] |
| PSA prepolymer | — | 286 | 2 | 1810, 1740 |
| PSA | 78 | 8392 | 45 | 1810, 1740 |

The IR data are characteristic of anhydride bonds.

Infrared spectra were collected on a Nicolet Magna-IR 750 spectrometer (Series II). Samples were prepared for analysis by film casting a solution of polymer in chloroform onto NaCl plates. Both polymers exhibited IR absorption peaks at 1810 and 1740 cm⁻¹, which are characteristic of aliphatic anhydrides.

PSA and griseofulvin particle samples were analyzed with a scanning electron microscope (SEM) model ISI-SX-30 to determine particle shape and morphology. Samples were prepared for SEM analysis by mounting a piece of double-stick carbon tape on an aluminum stub and then placing a portion of the sample on the tape. The samples were sputter coated with gold and then imaged.

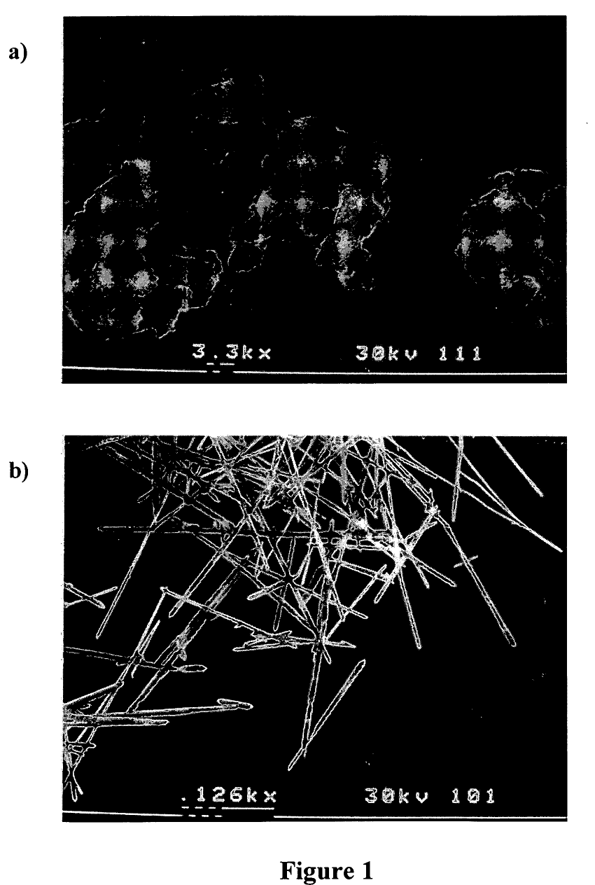

The morphology and crystal habit of PCA processed PSA-griseofulvin particles were examined as a function of the mass ratio PSA/griseofulvin in the feed, and the total solids concentration in the feed. Bulk griseofulvin obtained from the reagent bottle (unprocessed), and griseofulvin that had been crystallized under the same conditions as the drug-polymer mixtures were initially compared. FIG. 1a shows SEM micrographs of bulk griseofulvin unprocessed from the reagent bottle and FIG. 1b shows griseofulvin crystallized using the methods of the present invention but in the absence of a crystal growth inhibitor. As shown in FIG. 1b, griseofulvin crystallizes as long acicular needles in the absence of any PSA. The lengths of the crystals are approximately an order of magnitude larger than the widths. This is consistent with the published griseofulvin crystal habits produced when griseofulvin was crystallized from methylene chloride using compressed carbon dioxide as an antisolvent. SEM micrographs of pure PSA particles, and PSA-griseofulvin particles produced with mass ratios of 5:1 and 1:39 in which the total solids concentration in the feed to the injector was 0.75 wt % showed that the PSA particles are generally spherical in shape with smooth surfaces, and are slightly agglomerated. In contrast, PSA-griseofulvin particles produced with a mass ratio of 5:1 in the feed exhibit a different morphology relative to the pure polymer. These particles have a ‘jagged’ morphology with smooth surfaces, indicating that it is likely griseofulvin particles nucleated and began to grow with the characteristic acicular crystal habit, followed by precipitation of the polymer, which subsequently deposited as thin layer on the growing crystals. Decreasing the amount of PSA in the feed steam, with a concomitant increase in the amount of griseofulvin, had a profound effect on the morphology of the particles. The particle morphology switched from being dominated by the polymer to being dominated by griseofulvin, having well defined crystal faces. The change in griseofulvin crystal habit from acicular to bipyramidal is striking. The length of the longest axis of the griseofulvin crystals decreased by at least an order of magnitude between the acicular and bipyramidal habits. The crystals have well defined faces, and appear to be symmetrical. The mechanism responsible for the habit modification is believed to be the selective adsorption of PSA to the fastest growing crystal face of griseofulvin, which acts to inhibit growth, producing a more equant shaped particle.

Representative micrographs of PSA-griseofulvin particles produced with PSA-griseofulvin mass ratios of 5:1 and 1:39 in the feed, and a total solids concentration of 1.5 wt %, show the PSA particles similar to those produced with a feed concentration of 0.75 wt %. The microparticles are slightly agglomerated, and have smooth surfaces. Griseofulvin crystals with the same bipyramidal habit were produced when the total solids concentration was increased to 1.5 wt %, and the mass ratio of PSA/griseofulvin was 1:39. However, both large (>50 μm in length) and small (about 1-5 μm in length) crystals were observed.

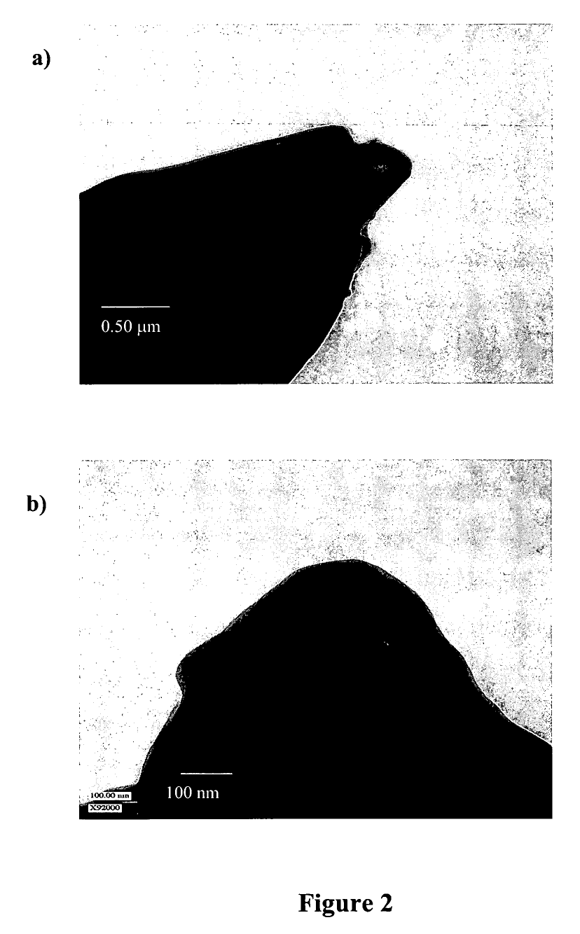

Several additional probe experiments were completed in order to investigate the concentration effectiveness of the growth inhibitor on the crystal habit of griseofulvin, and to elucidate the operating mechanism behind the growth inhibition. For this purpose, experiments were conducted with PSA/griseofulvin ratios of 1:1, 1:5, and 1:19, and a total feed concentration of 0.75 wt % was used. PSA and griseofulvin particles were seen to be physically segregated, however, as can been seen in FIGS. 2a and 2b, PSA microparticles can been seen to preferentially adsorb to the ‘tips’ of the bipyramidal griseofulvin crystals. Decreasing the PSA content in the feed resulted in a progressive decrease in the amount of PSA attached to the griseofulvin crystals; however, the crystals still maintained the distinct bipyramidal habit. The PSA particles were slightly agglomerated, and have a diameter of about 1 μm. In addition, the top faces of the bipyramidal crystals appeared to be rough, and PSA microparticles appeared to be fused to either end of the bipyramids. SEM images of crystals produced with a PSA/griseofulvin mass ratio of 1:19 showed that the primary bipyramial griseofulvin particles are similar to those produced using a lower amount of PSA in the feedstream, but agglomerates with a poorly defined shape were also seen. The agglomerates may have formed by the collision of two or more bipyramids, which were then fused together and coated by precipitating polymer.

An additional probe experiment was performed to determine the effect of varying the molecular weight of growth inhibitor (PSA) on griseofulvin crystal habit. The polymer solubility, molecular weight, and the number and type of functional groups within the polymer backbone are all expected to be important in controlling the effectives of the growth inhibitor. Micrographs of the PSA-griseofulvin particles produced when griseofulvin was crystallized in the presence of the PSA prepolymer with a feed concentration of 0.9 wt %, and a mass ratio of PSA prepolymer:griseofulvin of 1:1.2 showed a mixture of the two crystals habits (i.e. acicular and bipyramidal) were produced. In addition, the crystals appeared slightly agglomerated, with a thin, ‘tacky’ coating of polymer on the crystal faces. In contrast to the particles produced with a PSA/griseofulvin mass ratio of 1:1, where a physical mixture of both polymer and griseofulvin particles were produced, no separate polymer particles were observed with the PSA-prepolymer. It is possible the PSA-prepolymer was plasticized by the compressed CO₂, which prohibited the polymer from forming distinct microparticles, and therefore depositing as a thin film on the griseofulvin crystals.

A few samples were analyzed with a Transmission Electron Microscope (TEM) (Phillips, Model CM-10) to identify regions on the griseofulvin crystals where poly (sebacic anhydride) had selectively adsorbed. Samples were prepared for TEM analysis by placing small (<1 mg) amount of powder on a copper EM grid. The EM grids had been coated with carbon and formvar prior to sample preparation. The TEM images provided additional evidence to support a selective growth inhibition mechanism by PSA. The images of griseofulvin crystals produced with a total solids concentration in the feed of 0.75 wt %, and a mass ratio of PSA/griseofulvin of 1:39 indicated a change in material density, and hence a change in material properties. A clear change in contrast was observed on the ‘tip’ of the griseofulvin crystals. Doubling the feed concentration to 1.5 wt % while maintaining a constant mass ratio of PSA/griseofulvin of 1:39 gave a similar result. A thin film of PSA selectively adsorbed to the tips of the griseofulvin crystals, with a thickness of approximately 20 to 50 nm.

Particle Size Distributions (PSD)

Number weighted particle size distributions were measured using an Aerosizer (DSP model 3325, TSI Inc., St. Paul, Minn.) equipped with a dry powder dispersing system (Aero-Disperser, model 3230, TSI Inc., St. Paul, Minn.). The resolution of the Aerosizer, is 0.045 μm for a 1.0 μm diameter particle and 0.45 μm for a 10.0 μm diameter particle. Powdered samples were dispersed in the Aero-Disperser prior to being measured by the Aerosizer. To maximize the production of primary particles by the Aero-Disperser, the shear force in the disperser was set to 0.5 psi, the de-agglomeration setting was set to normal, and the feed rate was set to 5000 counts per second.

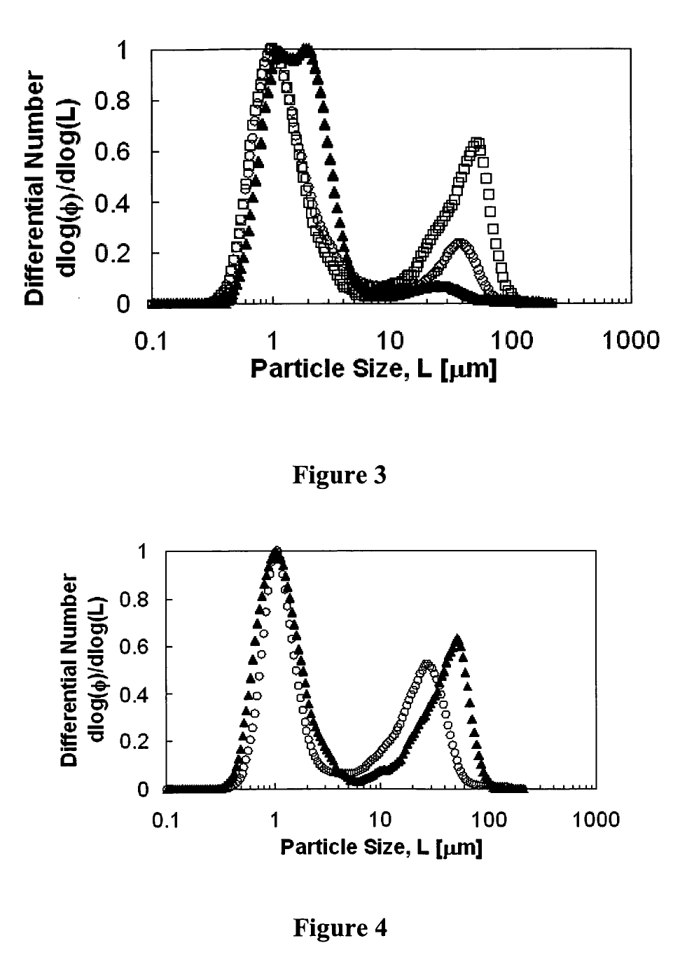

Particle size distributions were obtained at three different PSA-griseofulvin mass ratios. As shown in FIG. 3, all three particle size distributions are bimodal. The effect of decreasing the PSA concentration in the feed, with a concomitant increase in the griseofulvin concentration, resulted in a larger proportion of particles in the second mode between 10-100 μm. Particle size distributions for pure PSA, and PSA/griseofulvin mass ratios of 1:1 and 5:1 were also compared. Higher PSA concentrations did not result in smaller particle sizes in all cases. Intuitively, smaller particles would be expected following an increase in PSA concentration, since the polymer particles should dominate the distribution on a number weighted basis. As seen in FIG. 4, doubling the feed concentration from 0.75 to 1.5 wt % while maintaining a constant PSA/griseofulvin ratio of 1:39, resulted in a shift in the second mode of particles towards smaller size fractions. The shift in the particle size distribution is a result of enhanced nucleation over growth, resulting in the formation of a larger proportion of smaller particles.

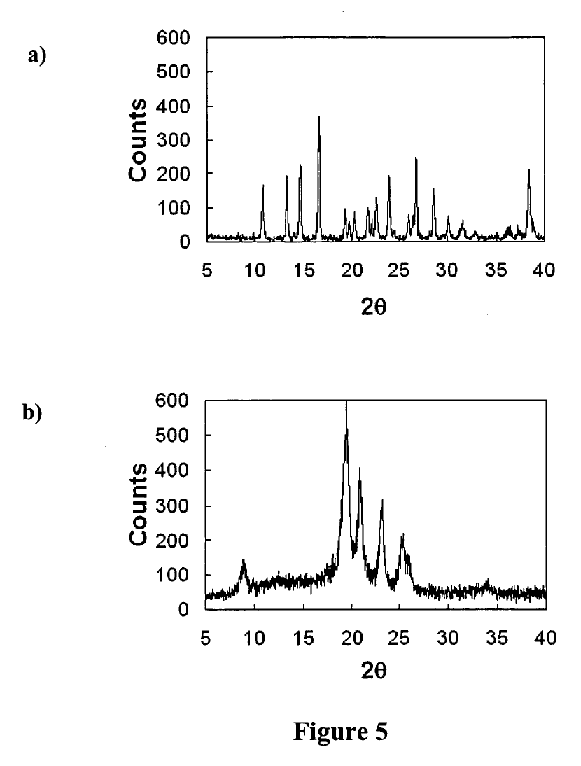

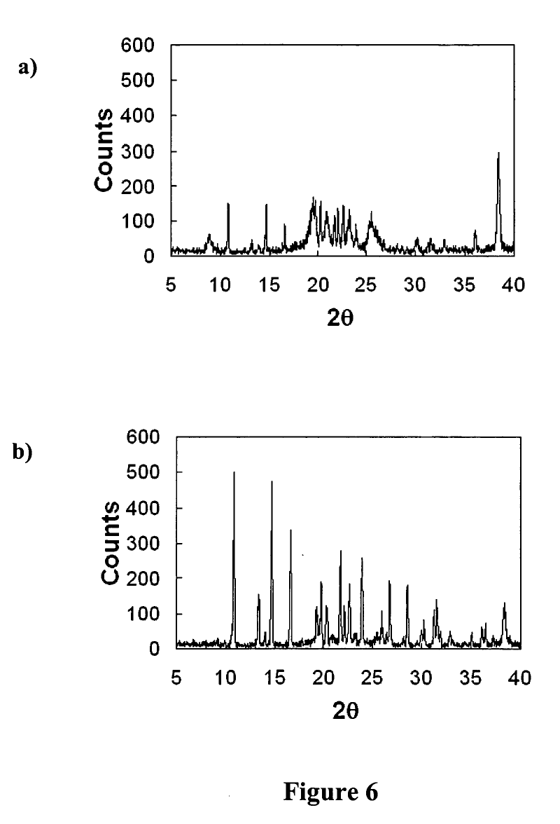

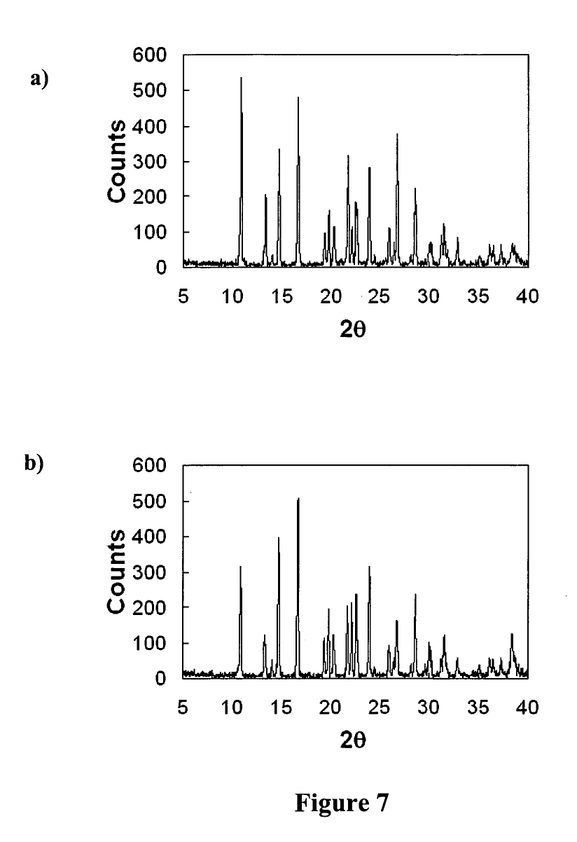

A Scintag diffractometer (Model No. Pad V) with a CuK radiation source, radiation wavelength of 1.5405 angstroms, was used to obtain the X-Ray Powder Diffraction (XRPD) pattern of selected samples. The current and voltage were set at 30.0 mA and 40 kV. Scans were conducted from 2 to 40° at a step width of 0.020 and a scan rate of 2°/min. Powder samples were prepared for analysis by compressing powder into an aluminum sample holder (8×11×1 mm) using a glass slide. XRPD spectra of bulk griseofulvin and synthesized PSA are shown in FIGS. 5a and 5b, respectively. Bulk griseofulvin exhibited numerous characteristic diffraction peaks between 10 and 27° 2θ, which indicated a high crystallinity. Pure PSA was semi-crystalline, and exhibited characteristic diffraction peaks between 15 and 30° 2θ0. PCA processed samples with a PSA/griseofulvin mass ratio of 1:1 and 1:5 are shown in FIGS. 6a and 6b, respectively. Characteristic diffraction peaks for both griseofulvin and PSA were observed in both spectra, although the crystalline peaks had less intensity relative to the pure component spectra. As can been seen in FIGS. 7a 7b, when the PSA/griseofulvin ratio was decreased to 1:19 and 1:39, the XRPD spectra of the samples were virtually identical to that of bulk griseofulvin, although the peak intensities were slightly higher. As shown in FIGS. 7a and 7b, no new peaks were observed in the diffraction patterns of the processed samples, which indicated griseofulvin did not transform to a new crystal form with a change in crystal habit. Also, no shifts in the griseofulvin peak positions were observed which demonstrates PSA was not incorporated into the griseofulvin crystal structure. A significant and systematic shift in the diffraction peak is expected when an additive is incorporated into the parent crystal structure. Furthermore, this spectra provides support for, and is consistent with, the crystal growth inhibition mechanism by PSA. Growth inhibiting additives that operate by a selective adsorption mechanism typically do not alter crystal structure, but rather alter only the crystal habit.

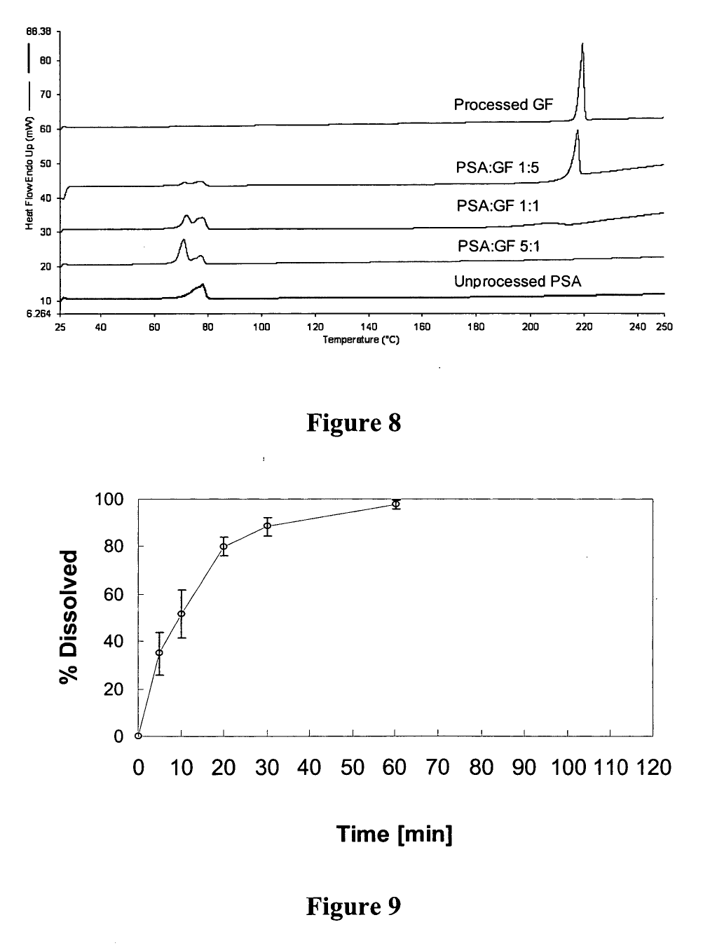

Differential Scanning Calorimetry (DSC) was performed on a DSC 7 calorimeter (Perkin-Elmer, Norwalk, Conn.) calibrated using pure indium (mp 156.6° C.) and zinc (mp 419.7° C.) standards. Powder samples (typically 3 mg) were accurately weighed into aluminum pans, and the pans were crimped with aluminum lids. All samples were heated from 25 to 250° C. with a 10° C./min heating rate. Thermal properties of the samples, i.e. melting points of the polymer and drug and enthalpies of fusion of the samples, were calculated using software provided with the instrument. FIG. 8 shows DSC thermograms for processed griseofulvin, unprocessed PSA, and PSA-griseofulvin samples with mass ratios of 1:5, 1:1, and 5:1. Pure griseofulvin exhibits a sharp melting endotherm at 219.6° C., with a ΔHf=112.58 J/g. Pure PSA exhibited a sharp endotherm at 78° C., corresponding to the melting of the crystalline regions of the polymer. The pre-transition prior to the sharp endotherm corresponding to the melting of the PSA is attributed to melting of the polymer folded chains. Endotherms corresponding to the melting of griseofulvin and PSA can be identified in all the spectra except the 5:1 PSA/griseofulvin sample. The fact distinct melting transitions are observed for both materials suggests griseofulvin and PSA have not formed a solid dispersion, and provides support for the growth inhibition mechanism proposed. PSA is expected to preferentially adsorb to a specific face of the griseofulvin crystals, and therefore will not form a solid dispersion. The absence of a peak corresponding to the melting of griseofulvin in the 5:1 sample is expected. This sample contained a low percentage (˜0.8 wt %) of griseofulvin in the powder, and this concentration is likely to be outside the detection limits of the instrument.

Surface Area Measurement

A Quantachrome Autosorb-IC (Boynton Beach, Fla.) was used to measure surface areas of the samples at 77 K. A known amount of powder (typically 500 mg) was loaded into a Quantachrome powder cell and outgassed for about 1 hr. Nitrogen was used as the adsorbate, and the equations derived by Brunauer, Emmett, and Teller (BET) were used by the software provided with the instrument to calculate the specific surface areas.

Powder Flow Properties

Bulk densities of selected samples were measured using a modified USP procedure. A mass of sample (M), typically 0.5 g, was passed through a No. 18 test sieve (ASTM USA Standard Test Sieve, Fischer Scientific) into a tared 10 mL graduated cylinder. The powder was carefully leveled and the unsettled apparent volume (Vₒ) was read to the nearest 0.05 mL. The bulk density (ρ_{b}) was then calculated using equation 1:

$${ρ}_{b} = \frac{M}{{V}_{o}} . \qquad ( 1 )$$

Each bulk density measurement was performed in triplicate, and the average value was reported. Tapped densities of selected samples were also obtained using a modified USP procedure. After the bulk density had been determined from the procedure described above, the graduate cylinder was mechanically tapped by raising the cylinder and allowing it to drop under its own weight using an apparatus that provided a fixed drop height of 14±2 mm. The nominal tap rate was 60 drops per minute. The cylinder was initially tapped 500 times and the tapped volume (V_{T}) was read to the nearest 0.05 mL. The procedure was then repeated, in increments of 500 taps, until the difference between succeeding measurements was less than 2%. The tapped density (ρₜ) was then calculated using equation 2:

$${ρ}_{t} = \frac{M}{{V}_{T}} . \qquad ( 2 )$$

Each tapped density measurement was performed in triplicate, and the average values for each sample is reported. Powder compressibility was computed from the bulk density and tapped density using equation 3:

$$\% Compressibility = ( \frac{{ρ}_{t} - {ρ}_{b}}{{ρ}_{t}} ) ⨯ 100. \qquad ( 3 )$$

Determination of Griseofulvin Content in the Powder Samples

The griseofulvin content of the powder samples were obtained by dissolving particles (about 2 mg) into methylene chloride, and then assaying the sample by UV spectroscopy at 292 nm. A standard calibration curve was constructed from known concentrations of griseofulvin in a PSA/methylene solution, and the griseofulvin concentration was determined by interpolation from the standard curve.

Powder flow properties, as represented by the bulk density, tapped density, and percent compressibility, were determined for selected samples and are shown in Table 2.0, in which GF is griseofluvin and the PSA:GF ratio was based on the original mass charged to the crystallizer. Absent values from the table indicate inadequate sample volume for the measurement technique.

TABLE 2

| Feed Concentration | PSA:GF | BET-Surface Area | Bulk Density | Trapped Density | Percent |
|---|---|---|---|---|---|
| [wt % solids] | Mass ratio | [m²/g] | [g/ml] | [g/ml] | Compressibility |
| 0.75 | 5:1 | — | 0.02 +/− 0.0002 | 0.04 +/− 0.0002 | 40 +/− 0.7 |
| 0.75 | 1:1 | 4.47 | 0.08 +/− 0.008 | 0.1 +/− 0.008 | 41 +/− 4 |
| 0.75 | 1:5 | 0.94 | — | — | — |
| 1.5 | 1:39 | — | 0.4 +/− 0.02 | 0.6 +/− 0.02 | 28 +/− 7 |
| NA | Bulk GF | 2.98 | 0.2 +/− 0.02 | 0.4 +/− 0.02 | 49 +/− 3 |

As shown by a percent compressibility of 28±7%, the 1:39 PSA-griseofulvin particles exhibited a significant enhancement in the flowability of the powder relative to bulk griseofulvin, which had a percent compressibility of 49±3%. Reasons for the enhanced flow properties of this material are due to the improved crystal habit, the larger average particle size, and the reduced number of fine particles in the sample. These properties are manifest in the bulk density, compressibility parameters, and the surface areas. It should be noted that powder flow properties were not obtained for griseofulvin samples that had an acicular habit. This testing procedure is invalid for acicular or plate-like crystal habits because particle attrition is likely to occur, and the PSD would be altered during the test

### Example 4

### Dissolution of Griseofulvin Having Modified Crystal Habit

A nonofficial USP dissolution study was performed using a modified version of the ‘tumbling method.’ The method involved placing a known mass of powder (equivalent of about 10 mg griseofulvin) within a Falcon tube, charging 50 mL of dissolution medium, mounting the tube to a Labquake shaker, and then rotating the shaker at 8 rpm for the duration of the study. The dissolution medium used consisted of a modified USP simulated gastric fluid which contained 4.0 wt % SDS. The simulated gastric fluid did not contain pepsin, since pepsin was found to interfere with the griseofulvin absorbance at 292 nm. Sink conditions were maintained by conducting the dissolution studies at 10% of the equilibrium solubility of griseofulvin in the dissolution medium. Samples (1.0 mL) were withdrawn at selected time points, filtered through a 0.2 mm filter, and then analyzed by UV spectrophotometry at 292 nm to determine the griseoftilvin content. Drug concentrations were determined by interpolation from standard curves constructed from known concentrations of griseofulvin in the dissolution medium. The temperature of the dissolution studies was maintained at 37±1° C. by placing the shaker apparatus inside an incubator. Dissolution profiles of the PSA-griseofulvin particles in a simulated gastric fluid which contained 4 wt % SDS are shown in FIG. 9. The mass ratio of PSA/griseofulvin in the feed was 1:19. All of the particles achieved 100% dissolution in one hour.

### Example 5

### Stability of Griseovulvin Having Modified Crystal Habit

A short term stability study was conducted at two International Conference on Harmonization (ICH) stability conditions, i.e. 25° C./60% relative humidity and 40° C./75% relative humidity. A thin layer of powder was placed in a 20 mL capped glass scintillation vial, and the vials were placed in a sealed desicator that was set to either 25 or 40° C., and to the desired relative humidity over the appropriate saturated salt solution³². Relative humidities of 60% and 75% were obtained using sodium bromide or sodium chloride, respectively. After initial storage (4 days), the samples were removed and characterized by XRPD. The samples were then returned to appropriate desicator and stored for another 19 days with the caps removed. Final sample characterization was by SEM and XRPD.

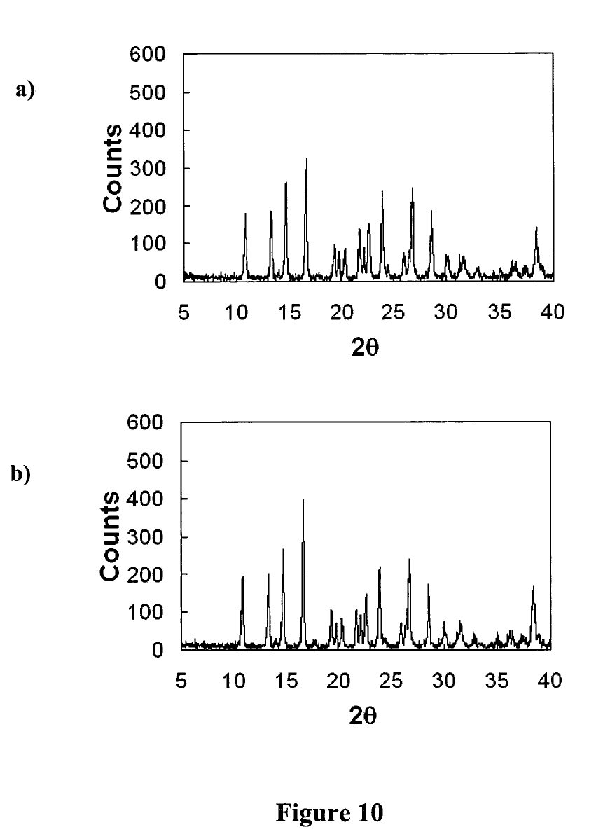

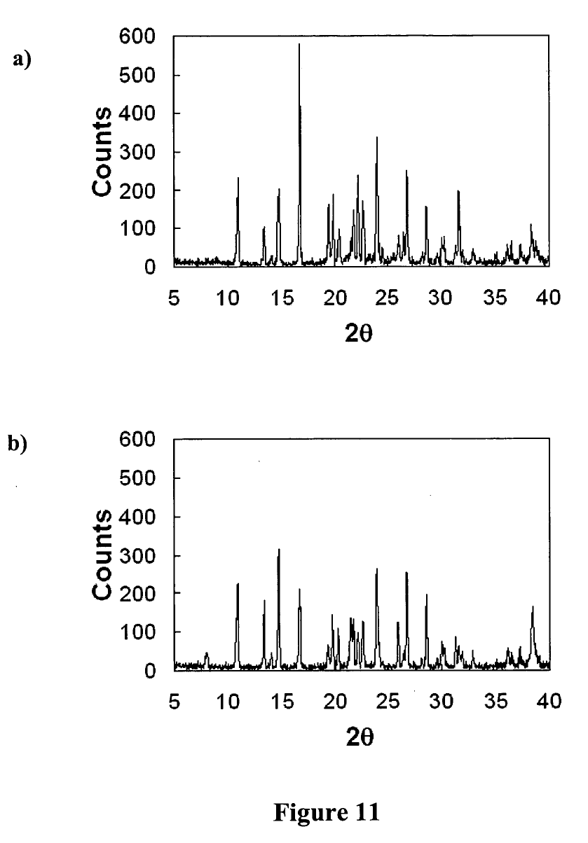

FIGS. 10a and 10b show the XRPD spectra of the bulk griseofulvin particles which were stored for 23 days at 25° C./60% RH and 40° C./75%, respectively. FIGS. 11a and 11b shows the XRPD spectra of PSA-griseofulvin particles (PSA/griseofulvin mass ratio of 1:5 in the feed) which were stored for 23 days at 25° C./60% RH and 40° C./75%, respectively. No new peaks were observed in the diffraction patterns for either sample after storage under these conditions, indicating griseofulvin did not undergo a phase change. Also, no shift in the peak positions was observed indicating griseofulvin did not interact with PSA to form a new polymorph. There was a slight change in the peak intensities in a few of the samples under the conditions investigated, and this may correspond to an increase in the amount of crystalline griseofulvin within the sample. However, the XRPD spectra of the samples showed no loss or gain of peaks indicating chemical and physical stability of the griseofulvin under the conditions investigated. A slight change in the morphology of PSA particles was observed, but this is expected since PSA is a hydrolytically unstable polymer. PSA is expected to degrade under the storage conditions tested, and this degradation may be responsible for the slight increase in peak intensity. Apparently PSA degradation does not affect the crystal structure of griseofulvin, as shown by the XRPD spectra.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A method comprising contacting a solvent comprising a compound and a crystal growth inhibitor, with an anti-solvent to extract the solvent from a co-precipitate of the compound and the crystal growth inhibitor.

2. The method of claim 1, wherein the crystal habit of the compound is modified without alteration of the crystal structure of the compound.

3. The method of claim 1, wherein the contacting is conducted in a confined mixing chamber.

4. The method of claim 1, wherein the co-precipitate is discharged into a particle collection vessel.

5. The method of claim 1, wherein the solvent is an organic solvent.

6. The method of claim 1, wherein the solvent is selected from the group consisting of methylene chloride, methanol, acetone, acetonitrile, methyl ethyl ketone, isopropanol, propanol, butanol, ether, benzene, hexane, hexanol, ethanol, cyclohexane, isooctane.

7. The method of claim 1, wherein the anti-solvent is a fluid selected from the group consisting of carbon dioxide, nitrogen, nitrous oxide, sulphur hexafluoride, xenon, ethane, ethylene, chlorotrifluoromethane, chlorodifluoromethane, dichloromethane, trifluoromethane, helium, neon, and mixtures thereof.

8. The method of claim 1, wherein the anti-solvent is at least one of a supercritical fluid and a near-supercritical fluid.

9. The method of claim 1, wherein the anti-solvent comprises a co-solvent selected from the group consisting of water, methanol, ethanol, isopropanol, acetone and combinations thereof.

10. The method of claim 1, wherein the compound is a pharmaceutical compound.

11. The method of claim 10, wherein the pharmaceutical compound is griseofulvin.

12. The method of claim 1, wherein the crystal growth inhibitor comprises a hydrophobic chemical.

13. The method of claim 1, wherein the crystal growth inhibitor comprises a polyanhydride.

14. The method of claim 1, wherein the crystal growth inhibitor comprises poly (sebacic anhydride).

15. The method of claim 1, wherein a mass ratio of the crystal growth inhibitor to the compound is equal to or less than about 1:1.

16. The method of claim 1, wherein a mass ratio of the crystal growth inhibitor to the compound is about 1:5.

17. The method of claim 1, wherein a total solids concentration of the solvent is between about 0.5 wt % and about 1.5 wt %.

18. The method of claim 1, wherein the contacting comprises an excess of the anti-solvent in relation to the solvent.

19. An article of manufacture comprising a co-precipitate of a crystalline compound and a crystal growth inhibitor.

20. A pharmaceutical formulation comprising a co-precipitate of a crystalline compound and a crystal growth inhibitor.

21. A pharmaceutical formulation comprising a co-precipitate of griseofulvin and poly (sebacic anhydride).

22. A co-precipitate of a crystalline compound and a crystal growth inhibitor comprising contacting a solvent comprising a compound and a crystal growth inhibitor, with an anti-solvent to extract the solvent from a co-precipitate of the compound and the crystal growth inhibitor.
